(19) United States
(12) Patent Application Publication
(10) Pub. No.: US 2013/0006091 A1
(43) Pub. Date: Jan. 3, 2013

(54) SYSTEM AND METHOD FOR A COMBINED MRI-PET IMAGER

(75) Inventors: Ravindra Mohan Manjeshwar, Glenville, NY (US); Thomas Kwok-Fah Foo, Clifton Park, NY (US)

(73) Assignee: GENERAL ELECTRIC COMPANY, SCHENECTADY, NY (US)

(21) Appl. No.: 13/175,040

(22) Filed: Jul. 1, 2011

(51) Int. Cl. A61B 5/055; A61B 6/03

(52) U.S. Cl. 600/411

(57) ABSTRACT

A combined magnetic resonance imager (MRI) and positron emission tomography (PET) imager and a method of performing combined MRI-PET imaging of an object is disclosed herein. The combined MRI-PET imager includes an MRI bore configured to perform MR imaging of the object. The MRI bore is sized so as to provide clearance between the MRI bore and the object within the MRI bore. The dedicated MRI-PET imager further includes a PET detector system is disposed outside the MRI bore to detect PET emissions from the object. The PET detector system includes at least one detector element retractably arranged exterior to the MRI bore. During the PET acquisition, the PET detector elements contract to a size so as to provide optimal clearance between the PET detectors and the object. During MRI acquisition, the PET detectors retract to allow the object to traverse into the MRI field of view

### BACKGROUND

The disclosure relates generally to magnetic resonance imaging (MRI) and positron emission tomography (PET) technologies. More specifically, the disclosure relates to a system and method that integrates PET and MRI technologies into a combined scanner capable of near-simultaneous PET and MRI imaging.

With increasing attention being given to imaging for traumatic brain injury, Alzheimer's disease, Parkinson's disease, epilepsy, and other forms of neurological disorders, a combined Magnetic resonance imaging (MRI) and positron emission tomography (PET) technology presents a significant leap forward in brain and neurological studies. The exceptional soft tissue contrast and high specificity of MRI together with PET' s excellent sensitivity in assessing physiological and metabolic state provide a precise combination of morphologic, functional, and metabolic information for diagnosis. Several combined MRI-PET imaging techniques have been proposed.

One approach has been to perform MRI and PET imaging on two separate scanners and later combine the two image information by image fusion methods for diagnosis. Although several approaches for sophisticated image fusion employing affine and deformable transformations have been developed, accurate spatial correlation of imaging data acquired sequentially with separate scanners is limited for several reasons. For example, patient repositioning causes differing section orientations, as well as variations in organ shape and position. Furthermore, the state-of-the-art registration algorithms are not able to register all deformations accurately and the confidence with which clinicians read the fused images may be compromised.

Improved data alignment may be achieved by hybrid systems enabling temporal and spatial co-registration of morphologic and functional data in a single examination and without repositioning the patient. Such hybrid systems have been developed wherein in a first type of system, a first scanner and a second scanner are connected to each other through a transport rail. A table capable of holding an examination target is provided on the transport rail, thus sequentially obtaining a PET image and an MRI image. However, such a system has several drawbacks, for example the object transport rail occupies a large amount of space, and significant time is spent in transporting an object from the first scanner to the second scanner through the transport rail.

In another configuration of hybrid MRI-PET systems the PET scanner, which is typically the smaller modality of the two, is placed inside the MRI bore. In such an arrangement, the PET scanner is exposed to a typically high magnetic field environment of the MRI which causes interference or interaction between the two systems in the form of electromagnetic interference (EMI) , affecting its performance. Also, the MRI data acquisition hardware, like RF coils, will attenuate the PET signal further reducing PET performance. While the above geometry may allow for simultaneous MR/PET imaging, the MRI imager is also rendered sub-optimal for brain imaging owing to the presence of the PET detectors within its field of view reducing the efficiency of the MR data acquisition system.

Accordingly, there exists a need for an integrated PET-MRI system that is dedicated to performing near-simultaneous MRI-PET imaging for a given target and addressing the aforementioned deficiencies.

### BRIEF DESCRIPTION

In accordance with one aspect, a dedicated magnetic resonance imager (MRI) and positron emission tomography (PET) imager for performing imaging of an object, like the brain, is provided. The dedicated MRI-PET imager includes an MRI bore configured to perform an MR imaging of the object. The combined MRI-PET imager further includes a PET detector system disposed outside the MRI bore to detect PET emissions from the object. The PET detector system includes at least one detector element retractably arranged exterior to the MRI bore

In accordance with another aspect, a combined magnetic resonance imager (MRI) and positron emission tomography (PET) imager for performing imaging of an object is provided. The combined MRI-PET imager includes an MRI bore configured to perform a dedicated MR imaging of the object. The MRI bore is sized so as to provide optimal clearance between the MRI bore and the object within the MRI bore. The dedicated MRI-PET imager further includes a PET detector system disposed outside the MRI bore to detect PET emissions from the object. The PET detector system includes at least one detector element retractably arranged exterior to the MRI bore.

In accordance with yet another aspect, a method of manufacturing a combined MRI-PET imager is provided. The method includes configuring an MRI bore to capture an MR image of an object positioned within the MRI bore. The method further includes disposing a PET detector outside the MRI bore to detect PET emissions from the object where the PET detector system includes at least one detector element retractably arranged exterior to the MRI bore.

In accordance with a further aspect, a method of performing combined MRI-PET imaging of an object in a combined MRI-PET imager is provided. The method includes retracting a detector element of a PET detector to provide clearance for the object to traverse into an MRI field of view (FOV). An MR image of the object being positioned into the MRI FOV is captured. The method further includes receiving the object within the PET detector FOV and re-positioning the PET detector elements to provide optimal clearance between the PET detector and the object. Subsequently, a PET image of the object within the PET detector FOV is captured.

### BRIEF DESCRIPTION OF THE DRAWINGS

These and other features, aspects, and advantages of the present system and method will become better understood when the following detailed description is read with reference to the accompanying drawings in which like characters represent like parts throughout the drawings, wherein:

FIG. 1 is a schematic representation of an exemplary embodiment of an MRI-PET imaging system.

FIG. 2 is a block diagram representation of an exemplary MRI-PET imaging system configuration in accordance with one embodiment.

FIG. 3 is a schematic representation of a configuration of the PET detector system in accordance with one embodiment.

FIG. 4 is a schematic representation of a configuration of the PET detector system in accordance with one embodiment.

FIG. 5 is a schematic representation of a configuration of the PET detector system in accordance with one embodiment.

FIG. 6 is a schematic representation of a configuration of the PET detector system in accordance with one embodiment.

FIG. 7 is a flow chart representing steps involved in an exemplary method of performing a combined MRI-PET imaging in accordance with one embodiment.

FIG. 8 is a schematic representation of a profile of the MRI-PET imaging system during one stage of operation in accordance with one embodiment.

FIG. 9 is a schematic representation of a profile of the MRI-PET imaging system during one stage of operation in accordance with one embodiment.

FIG. 10 is a schematic representation of a profile of the MRI-PET imaging system during one stage of operation in accordance with one embodiment.

FIG. 11 is a schematic representation of a profile of the MRI-PET imaging system during one stage of operation in accordance with one embodiment.

FIG. 12 is a block diagram representation of a perspective view of the MRI-PET imaging system during one stage of operation in accordance with one embodiment.

FIG. 13 is a block diagram representation of a perspective view of the MRI-PET imaging system during one stage of operation in accordance with one embodiment.

FIG. 14 is a block diagram representation of a perspective view of the MRI-PET imaging system during one stage of operation in accordance with another embodiment.

FIG. 15 is a block diagram representation of a perspective view of the MRI-PET imaging system during one stage of operation in accordance with another embodiment.

FIG. 16 is a flow chart representing steps involved in an exemplary method of manufacturing a combined MRI-PET imager in accordance with one embodiment.

### DETAILED DESCRIPTION

As described in detail below, embodiments of the present system provide a combined magnetic resonance imager (MRI) and positron emission tomography (PET) imager for performing imaging of an object and method for the same. According to an embodiment, a system geometry is described that is optimized for both dedicated MR and PET imaging of an object of interest. The proposed MRI-PET imager obtains near-simultaneous PET and MR images of the object of interest.

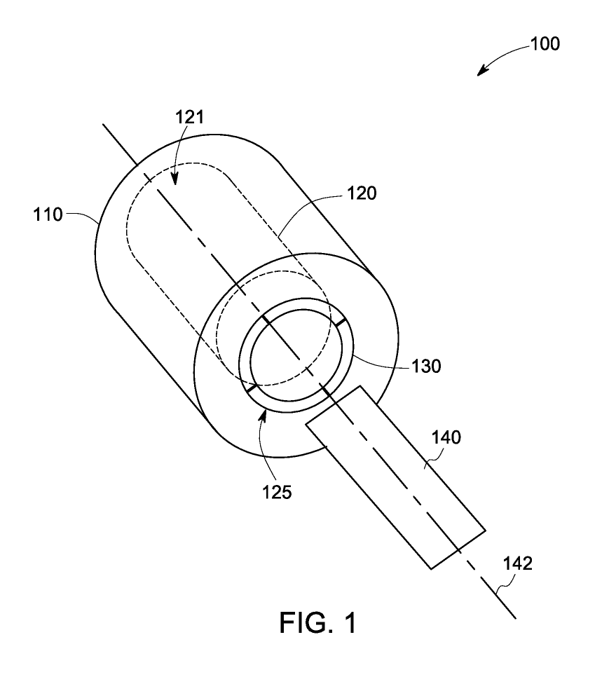

FIG. 1 is a schematic representation of an exemplary embodiment of a combined Positron Emission Tomography (PET)-Magnetic Resonance Imaging (MRI) imaging system 100. The combined MRI-PET imaging system 100 includes an MRI imager 110 having an MRI bore 120 for capturing MR images of an object (not shown), a PET imager 125 including a PET detector system 130 installed outside the MRI bore 120, and an object table 140 for transporting the object into the PET detector system and MRI bore 120. An example of the object includes, but is not limited to humans and animals as well as other objects in which it is beneficial to obtain soft tissue contrast and high specificity of MRI together with PET' s sensitivity in assessing physiological and metabolic states. The MRI bore 120 is configured such that an MRI imaging space 121 is formed through the center of the MRI bore 120, and the object table 140 is mounted to allow the object to move into the MRI imaging space 121 while the object is on the object table 140. The object table 140 may be slidably formed to transport the object in and out of the imaging space.

The MRI imager 110 includes a main magnet (not shown) having a hollow cylindrical geometry. The main magnet is the largest and outermost component in a combined MRI-PET imager 100. The main magnet may include, but is not limited to a permanent magnet, a resistive electromagnet, and a superconducting electromagnet. The main magnet generates a strong and uniform magnetic field B₀ during an MR imaging of the object. The MR image is typically acquired within a central region of the MRI imaging space 121, hereinafter referred to as MR field of view (FOV), along the main axis 142 of the primary magnet due to strong uniformity of the magnetic field in such region.

The PET detector system 130 includes one or more detector elements for detecting coincidence annihilation photons emitted from the object. In operation, the detector elements are arranged about the object and are configured to have at least a size such that the coincidence annihilation photons emitted in the direction of the detector elements from the object is detected. In the illustrated embodiment, the PET detector system 130 is constructed to have a circular geometry to facilitate alignment with the MRI bore 120. Specifically, the PET detector system 130 is placed proximate the bore 120 of MR main magnet generally along the main axis 142 of MR main magnet. In one example the PET detector system is placed outside the bore 120 and can be an integral part of the system, an add-on component or a separable component that can be deployed as needed.

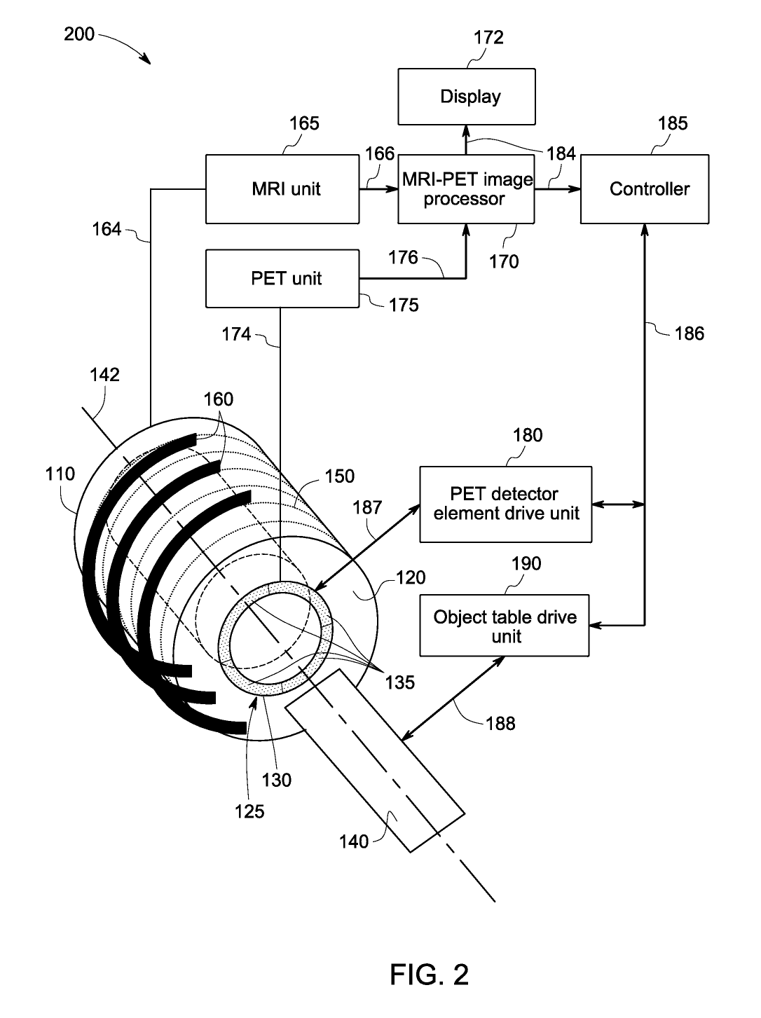

FIG. 2 is a block diagram representation of a combined MRI-PET imaging system 200 configuration in accordance with one embodiment. As illustrated, the MRI imager 110 includes a main field magnet formed of a set of magnetic field coils 160 and a high-frequency radio wave system formed of a set of radio-frequency (RF) coils 150 for generating HF excitation pulses and for detecting the emitted resonance signals.

The set of radio-frequency (RF) coils 150 sometimes referred to as an MR antenna is generally located within a central region of the magnetic field Bₒ produced by the magnetic field coils 160. These RF coils 150 may have at least two functions specifically, to transmit RF signals and to receive RF signals from an object during an MR imaging process. During the transmission of RF signals, RF coils 150 typically generate an RF pulse oscillating at the Larmor frequency of the spins, which excites the nuclei in the object to be imaged. During reception of the RF signals, the RF coils 150 detect the signals at the similar frequency emitted by the same nuclei during their “relaxation” to the original states. Note that the object being imaged is placed inside the bore encompassed by the RF coils 150, so that the object is within a central region 121 of the magnetic field B₀.

The image signals 164 captured and obtained by the MRI imager 110 are transmitted to an MRI unit 165, and further converted into images. The images are transmitted to a combined MRI-PET image processor 170 where the MR images 166 may be combined or mapped with PET images 176 detected by the PET detectors. The MR and PET images 184 may be displayed on a display 172.

The PET detector system 130 includes one or more detector elements 135. There are various configurations for the arrangement of the detector elements 135. In one embodiment, the detector elements 135 may be arranged in the form of a ring that surrounds the object. Such a PET imaging system is also sometimes referred to as a stationary block ring system. In another embodiment, the PET imager 125 may, for example, also include two, four or six flat detectors. Furthermore, it is possible to extend both transaxial and axial fields of view (FOV) of the PET detector system 130 outside the MRI bore 120.

The data for a number of PET tomograms are typically acquired sequentially in time by displacing the PET detector 130 in an axial direction along the main axis 142 in stepwise fashion. It is also possible to use a smaller number of large-area position sensitive detector elements in a polygonal arrangement. Moreover, it is possible to use a ring shaped detector that only partially configured with detector elements. In the example for the ring detector, the detector elements 135 are rotated about the object in order to acquire the requisite measured data. Such a PET imaging system is also sometimes referred to as a rotating block ring system.

The PET detector elements 135 are typically formed of scintillation crystals arranged in an array and coupled to a photo-sensor. The signal processing performed by the PET imager 125 in accordance with one example is described herein. The scintillation crystals stop the annihilation photons emitted from the object and convert them into light scintillation pulses. The scintillation pulses produced by the scintillation crystals in the PET detector elements 135 is transmitted to a photo sensor (not shown), and is further converted into charge signals. In a particular embodiment, the photo sensor is a photo-multiplier tube. In another embodiment, the photos sensor is a semiconductor photo sensor like an Avalanche Photo-Diode (APD) or a Solid State Photo-Multiplier (SSPM). The charge signals output from the photo sensor are transmitted to a signal amplification circuit within a PET unit 175. Fine charge signals are amplified while passing through the signal amplification circuit, and the amplified signals are encoded/decoded into the energy, interaction time and position information while passing through the PET unit 175. The detected signals from the PET emissions are converted into images with functional information through a process called tomographic image reconstruction. The reconstructed images 174 are transmitted to the combined MRI-PET image processor 170 and are further combined into a single image by the image processor 170. Thus, a combined image into which an anatomical image and a functional image are combined is obtained. Furthermore, the combined image processor 170 may selectively combine respective images into a single image or separate a single image into respective images. The processed MR and PET images may also be stored in a non-volatile or volatile storage medium (not shown).

Although not shown in detail in FIG. 2, the MRI imager 110 also includes a set of gradient coils, which generate field gradients onto the main field B₀ in the x, y, and z directions. The field gradients are used to encode the distance information in the space where the subject is located. The PET imager 125 also includes electronics (e.g., associated preamplifiers) and other metal components (e.g., shielding enclosures).

FIG. 2 presents a perspective view of an integrated PET-MRI imager illustrating the spatial relationships between the components in accordance with one embodiment. Note that the components in this example are generally constructed concentrically or nearly concentrically with respect to the main axis 142 of main magnet 160. Although it is desirable to have the components arranged in such a manner, one or more components may be slightly off-axis.

In an exemplary embodiment, the PET detector elements 135 are configured to be movable in a transaxial direction about the axis 142 of the MRI bore 120. As used herein, the term ‘transaxial direction’ refers to the direction along the circular circumference on which the detector elements are fastened. In another embodiment, the MR antenna 150 may be installed such that it translates in an axial direction 142 within the MRI bore 120. As referred herein, the term ‘axial direction’ is the direction along axis along which an object table is arranged, which is also the axis 142 of the main MR magnet.

The displacement of the PET detector system 130 in a transaxial direction, and the rotation, required if appropriate, about the object are performed according to an exemplary embodiment with the aid of a PET detector element drive unit 180. The PET detector element drive unit 180 may employ fluid hydraulics or compressed air hydraulics and operates according to the control signals received from a controller 185. In a particular embodiment, the MR antenna 150 and the PET detector system 130 may have a common drive unit 180 or have separate drive units. The common drive unit and the separate drive units enable the independent movement of the MR antenna 150 and the PET detector system 130 in an axial and transaxial direction respectively. The controller 185 provides control signals 186 based at least partly on the MRI, PET image data 184 received from the MRI-PET image processor 170. The drive control signal 187 for controlling the movement of the PET detector system 130 may be transmitted from the drive unit 180 to the PET detector system 130 with the aid of Bowden cables, push rods, toothed belts, or any other mechanical or electronic means.

In another aspect, the combined MRI-PET system 200 may further include a drive unit 190 for the object table 140. Again, the object table drive unit 190 operates according to the control signals 186 received from the controller 185. The drive control signal 188 for controlling the movement of the object table is provided by the object table drive unit 190. The PET detector element drive unit 180 and the object table drive unit 190 may be coupled to suitable position sensors or motion sensors (not shown) for sensing a motion or position of the PET detector system 130 and object table 140. Based on the received sensor signals, the controller 185 directs the drive units 180, 190 so that the movement of the object table 140 is synchronized with a movement of the elements of the PET detector system 130. For example, the elements of the PET detector system 130 may be configured to extend from an imaging position to an open position in tandem with the object table 140 advancing towards the MRI bore 120. Similarly, the elements of the PET detector system 130 may be configured to retract back to the imaging position from the open position in tandem with the object table 140 receding away from the MRI bore 120. As used herein, the PET detector system 130 in one example operates in at least two distinct positions, namely an imaging position and an open position. The open position refers to the removal of the PET detector from the MRI bore opening thereby allowing easy entry of an object on the table 140 to gain entry to the MRI imager 120. The open position can be the retraction of the PET detector elements 135 or the removal of the PET detector system 130. The imaging position for the PET detector system 130 refers to the position of the PET detector that allows for imaging.

According to another embodiment, the combined MRI-PET imager 200 is adapted to performing dedicated imaging of an object. The proposed dedicated MRI-PET imager 200 is configured with the PET detector system 130 positioned immediately outside or otherwise proximate the bore 120 of the MRI imager 110. The MRI bore 120 of the dedicated MRI-PET imager in one example is sized to a dimension selected according to a dimension of the object of interest, a standard range of such dimensions or a pre-specified dimension. In one example, assuming that the object has at least two sections of varying dimensions, where the second section of the object has a larger volume than the first section of the object, the object of interest may be the first section of the object. In order to perform dedicated imaging of the object of interest, at the time of manufacture, the MRI bore is sized so as to provide a minimum required clearance between the object of interest and the circumference of the bore 120. In one embodiment, the minimal spacing required may be between about 10 and about 15 cms. It should be noted that sizing of the MRI bore refers to sizing the RF coils and the gradient coils that define the bore.

Furthermore, the PET detector 130 is sized so as to have a PET imaging space when operating to perform imaging using the PET detectors. The sizing the PET detector system 130 refers to distributing the detector elements 135 of the PET detector system 130 around the object at an appropriate distance for the required imaging. Additionally, in one example the PET detector system 130 is configured to dynamically extend radially or transaxially outward from the object to provide clearance for the object that may be translated into the MRI bore 120. As used herein, the term “dynamically” is characterized by an action performed at any given instant of time. Also, as used herein, the direction from the center of the PET detector to the circumference on which the detector elements are fastened is the radial direction.

According to one example, subsequent to MR imaging of the object within the MRI bore 120, a first section of the object is moved out of the MRI bore 120 and into the PET imaging space thereby moving a second section out of the PET imaging space. The PET detector system 130 is configured to retract back to its original imaging/closed position for PET imaging the first section of the object. An object table may be used for moving the object between the fields-of-view of the MR and PET imagers for MR and PET imaging, respectively.

Consider one example, wherein the MRI-PET imager 200 is dedicated to imaging the head of a human such that the MRI bore 120 and the PET detector system 130 are both sized to a dimension that provides a minimum required clearance to accommodate the head. When the object table 140 advances the patient head into the MR FOV for MR imaging, the PET detector system 130 retracts to an open position to clear the patient shoulders. During PET imaging, the PET detector system 130 retracts back to an imaging position for imaging the patient head such that the patient table 140 is drawn back to move the patient head out of the MRI FOV into the PET FOV. In the case of a segmented PET detector system, the segments contract to form a tight bore, such as with seamless (or with small seams) detectors to permit the optimal PET imaging. The dedicated MRI-PET imager described herein provides for optimal, high sensitive and high resolution PET imaging. In another embodiment the PET detector is removable and is removed when performing the MRI imaging and replaced when performing the PET imaging.

The combined MRI-PET imager of at least one embodiment includes a PET detector system 130 that may be displaced or extended in a radial, linear, or circumferential direction about the main axis 142 of the main magnet. Such movable configuration of PET detector system 130 allows for dynamically altering a transaxial field of view (FOV) of the PET detector system 130. In a particular embodiment for performing a dedicated imaging of an object, the PET detector system 130 is configured to extend and retract in order to provide clearance for a specific section of the object and for dynamically adjusting the PET FOV.

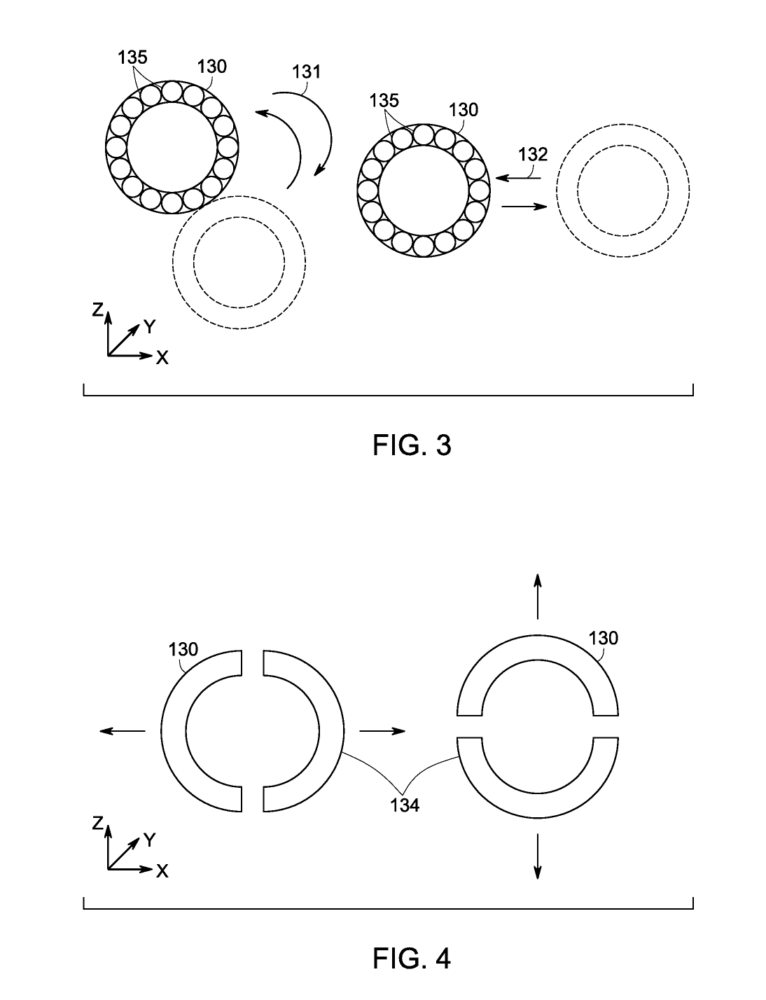

The PET detector system 130 may have several different configurations as shown in FIGS. 3-6. For example as shown in FIG. 3, the PET detector elements 135 are configured as a detector ring 130 occupied with one or more PET detector elements 135 that surround an object (not shown). The PET detector system 130 may be fully or partially occupied with detector elements 135 whose number and distribution on the detector ring are selected such that the acquisition of the measured data that is required for producing the PET images of one or more layers of the object is possible with or without rotation of the detector system 130. In one orientation, the detector ring 130 is slidably coupled to the MRI imager and the MRI bore. As shown, the detector ring 130 may slide linearly (along X, Y, Z directions), as illustrated by arrows 132 or circumferentially about the main axis of the main magnet, as illustrated by arrows 131. In one arrangement, the detector ring 130 may be configured to slide along linear or radial sliding guides by electromechanical means, such as a motor and gear assembly. In another arrangement, the detector ring 130 may be configured to slide along linear sliding guides by mechanical means such as pneumatic or hydraulic actuators. Further, the detector ring 130 may be configured to slide in tandem with a movement of the object table by several coupling means such as using rigid link between the sliders with pivot points on the sliders, joining the sliders with belts, chains or guides, and mechanically joining the sliders using gears.

In the case of a PET detector system 130 that is occupied only partly with detector elements, the detector ring 130 is rotated about the object until the measured data for a first complete PET image of the relevant axial field-of-view has been acquired. Subsequently, the object table 140 may slidably transport the object in the axial direction, which is to say in the direction in which the object is supported, that runs perpendicular to the circular circumference on which the detector elements are arranged. After the transportation of the object as far as the next axial field of view of the object, the next PET image may be recorded by rotating the PET detector system 130. The PET detector system 130 may be rotated by any electrical, electromechanical, or mechanical means. A number of PET tomograms are continuously acquired during such various displacement and rotation steps, thereby reducing the overall measuring time.

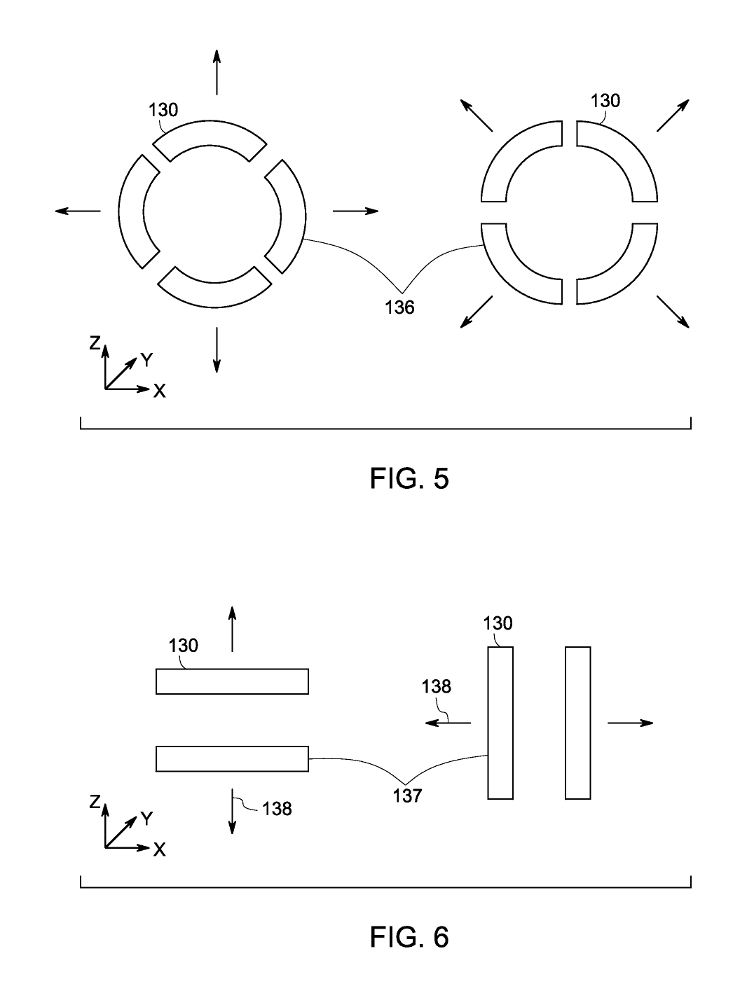

The PET detector system 130 may also be divided into two or more segments as shown in FIGS. 4-5, specifically segments 134 in FIG. 4 and segments 136 in FIG. 5. The segments 134 and 136 of the PET detector system 130 are configured to extend or contract radially about the main axis of the main magnet (not shown).

In another exemplary orientation as shown in FIG. 6, the PET detector system 130 may be configured as two separate detector plates 137 that are arranged parallel to one another on opposite sides of the object and that may be extended in the direction of the arrows 138 or may be rotated in a transaxial direction about the object (not shown) for the purpose of complete data acquisition. In yet another embodiment, the PET detector system 130 may include more than two detector plates so as to surround the object completely in a polygonal arrangement, the number of the detector plates being even or odd in number. Such PET detector plates are usually referred to as continuous detector panels.

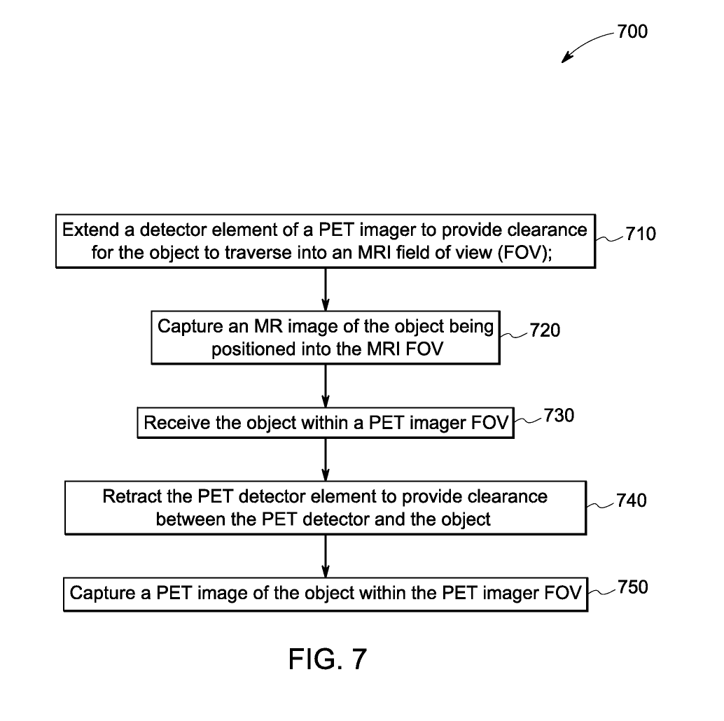

A method by which the combined MRI-PET imager constructed as described above is operated will be described below with reference to the flowchart 700 shown in FIG. 7.

Broadly speaking, for the purpose of imaging an object in the combined MRI-PET imager, the object to be imaged is placed on an object table and moved between the fields-of-view of the MR and PET imagers for MR and PET imaging, respectively. Subsequently, the images produced are superimposed in a processor, thus combining the high spatial resolution of an MRI with the functional information from PET.

In more detail, the method 700 in one embodiment includes preparing the object for PET imaging by introducing a tracking agent such as a radiopharmaceutical by injection or inhalation. The object is positioned on the object table for locating the object within the MRI and PET FOVs. When the object table is advanced towards the PET detector system, the PET detectors are extended or removed in step 710 from an imaging position to an open position to provide clearance for the object to traverse into the MRI FOV. An MR image of the object within the MRI FOV is further captured in step 720. Subsequent to MR imaging the object, the object table is receded such that the object is traversed out of the MRI bore and positioned within the PET detector FOV. After receiving the object within the PET field of view in step 730, the PET detector is retracted back to the imaging position so as to provide optimal clearance between the PET detectors and the object for PET imaging in step 740. In a particular embodiment, an aperture defined by the at least one detector element by retracting the detector element is controlled to provide optimal clearance. Subsequently, a PET image of the object within the PET imager FOV is captured in step 750. In one embodiment, the object is traversed through the combined MRI-PET imager in tandem with the extending and retracting of the PET detector element. The two images are further combined using signal processing methods. In should be noted that the above steps may be performed in any preferred order, for example, the PET imaging of the object may be performed prior to MR imaging of the object. Since it is possible to perform MR imaging and PET imaging within a time difference of about a few seconds to about a few minutes, the combined MRI-PET imaging is considered to be near-simultaneous.

It should be noted that the steps of the method described above may be adapted to a method of performing a dedicated imaging of an object using the dedicated MRI-PET imager in which the MRI bore is configured to accommodate only an object of a specific dimension. In other words, the MRI bore is sized so as to accommodate only a certain object or part of an object such as the head of a human or animal Similarly, the PET detector system is configured such that the PET detectors may provide clearance for an object, as the object is being advanced into the MRI bore, only in an extended or displaced position. For example, the PET detectors may provide clearance for a patient's shoulder only in the extended or displaced position thereby allowing the patient's head to be positioned within the MRI FOV.

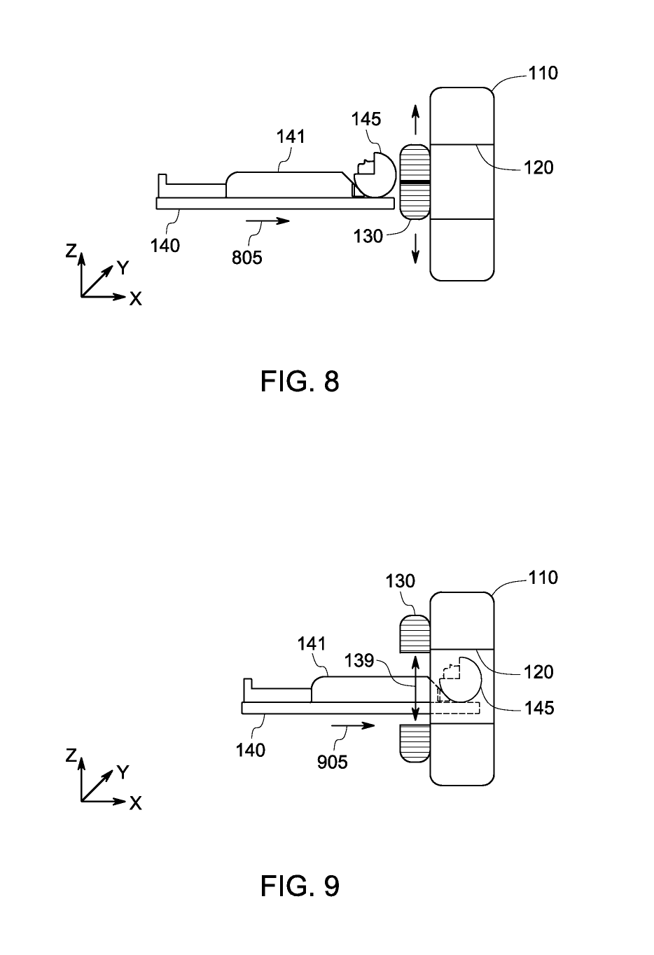

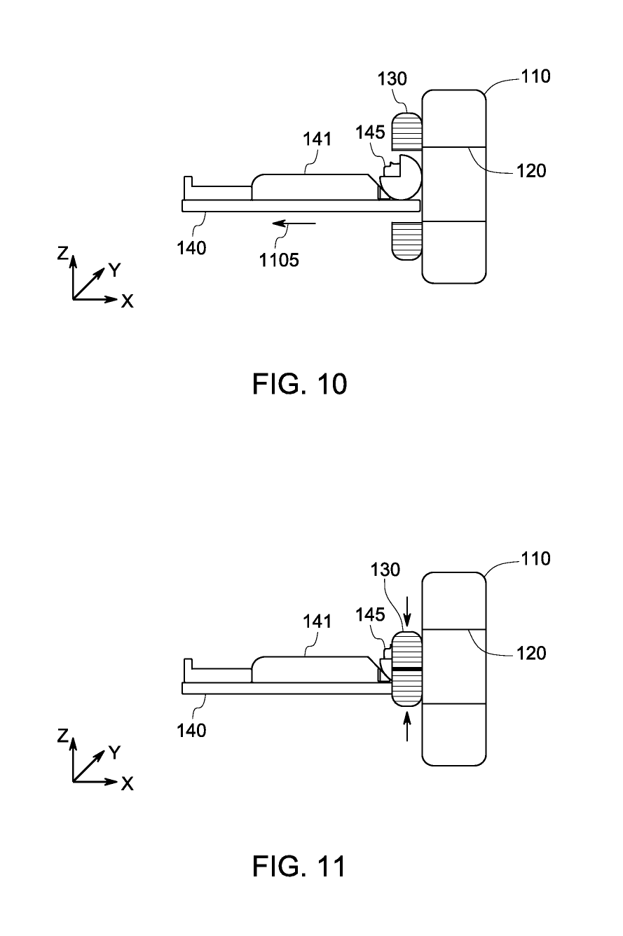

FIGS. 8-11 show a schematic representation of a profile of the MRI-PET imaging system 100 (FIG. 1) during various stages of operation in accordance with one embodiment. It should be noted that the PET detector system 130 illustrated in FIG. 8 may be configured according to any of the orientations illustrated in FIGS. 3-6. As the object, e.g. a patient 141, is advanced towards the combined MRI-PET imager along the direction of the arrow 805, the PET detector system 130 extends outwardly from the patient along any of the directions (X, Y, and Z) radially. For example, assuming that the PET detector system 130 is configured as a segmented ring detector having two segments, each of the two segments may extend along one of (X,-X), (Y,-Y), or (Z,-Z) directions. As shown in FIG. 9, the PET detector system 130 provides clearance 139 to the patient's shoulder so that the patient's head 145 is further advanced through the PET detector 130 into the MRI bore 120 along the direction of the arrow 905. After an MR image of the patient's head 145 is captured, the patient is moved out of the MRI bore 120 along the direction of the arrow 1105 as shown in FIG. 10 such that the patient's head 145 is positioned within the PET FOV. The PET detector segments which are now in an extended position are retracted back to an imaging position towards the patient's head 145 as shown in FIG. 11. It should be noted that the retracting of the PET detector system 130 to an imaging position also provides the minimum required clearance between the PET detector system 130 and the patient's head 145 for obtaining optimal PET images of the patient's brain.

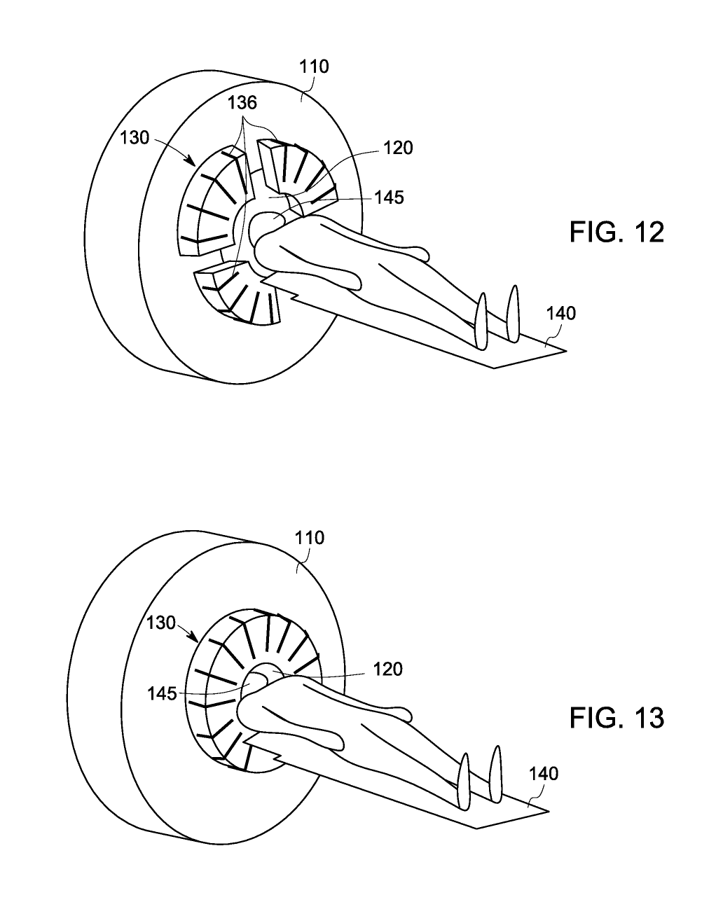

FIGS. 12-13 show a perspective view of the combined MRI-PET imager 100 (FIG. 1) with the PET detector system 130 configured as a detector ring having 4 segments 136. The PET detector system 130 extends and retracts between an open position as illustrated in FIG. 12 and a closed position as illustrated in FIG. 13.

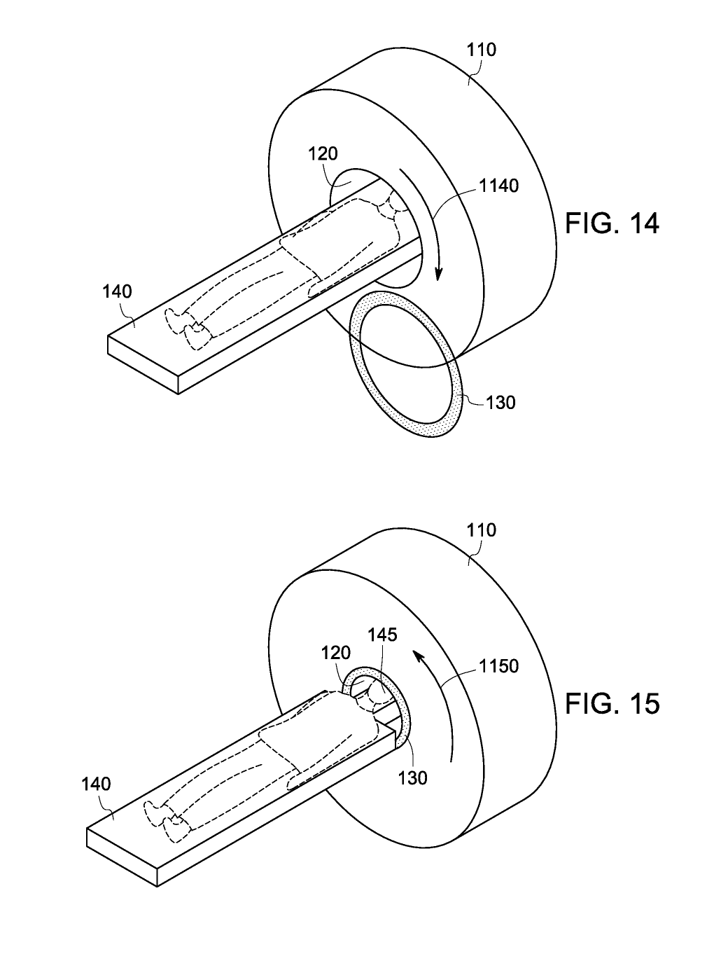

FIGS. 14-15 show a perspective view of the combined MRI-PET imager 100 (FIG. 1) with the PET detector system 130 configured to slide circumferentially between an open position, as illustrated in FIG. 14 and a closed imaging position as depicted in FIG. 15 about the main axis, along the direction of the arrows 1140, 1150. In operation, the PET detector system 130 may be positioned in the open position to provide clearance for an object 145 to traverse into the MRI bore 120. Subsequent to performing MR imaging of the object 145, the object 145 is drawn out of the MRI bore 120 so as to provide clearance for the PET detector system 130 to slide to the imaging position shown in FIG. 15. The object 145 is further advanced towards the PET detector system 130 and positioned within the PET FOV. Subsequently, a PET imaging of the object 145 is performed. It should be noted that the PET detector system 130 may have either the open/extended position (FIG. 14) or the closed/imaging position (FIG. 15) as the default position with the order of steps being altered accordingly. For example, in the case where the default position of the PET detector system 130 is the closed imaging position as shown in FIG. 15, the PET imaging of the object 145 is performed after which the object 145 is drawn out of the PET detector's FOV, followed by a sliding of the PET detector system 130 to the open position as shown in FIG. 14 and moving the object 145 into the MRI bore 120 for MRI imaging.

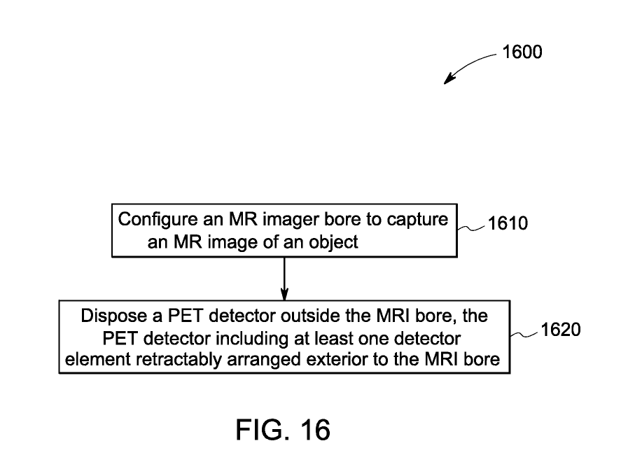

FIG. 16 is a flow chart representing steps involved in an exemplary method 1600 of manufacturing a combined MRI-PET imager in accordance with an embodiment. The method 1600 includes configuring an MRI imager bore to capture an MR image of an object in step 1610. In one embodiment, the MRI bore is sized so as to provide optimum clearance between the MRI detector elements such as the RF antennas and the object. For example, the optimum clearance between the object and the head for obtaining high quality MR and PET images is in the range of about 10 to about 15 cms in circumference. Such a configuration of the MRI bore allows for capturing accurate and high quality images with reduced artifacts. As is understood, the customization of the MRI bore size to a specific dimension allows MR imaging of objects having similar or smaller dimensions unless the detector elements of the MRI imager are adjustable to alter the circumference defined by the detector elements about the object. For example, the MRI bore may be sized to measure a circumference of about 40 to about 60 cms for accommodating an average human head. Since the MRI bore is customized for a human head, the clearance between the head and the bore is optimal making it is possible to obtain high resolution images. On the contrary, if the MRI image bore is sized to accommodate the human body with a bore circumference of 160 cms, then an MR imaging of the head may be suboptimal with a large clearance between the head and the circumference of the bore. A PET detector system 130 is disposed in step 1620 outside the MRI bore where the PET detector system includes one or more detector elements retractably arranged exterior to the bore. In an embodiment, the MRI-PET imager is dedicated to performing combined imaging for a specific object, e.g., a human head for brain imaging.

Further, the PET detector system is configured according to any of the exemplary orientations shown in FIGS. 3-6. For example, the PET detector system may be configured as a segmented ring as shown in FIG. 5. In this configuration, the four segments of the PET detector system may be configured to extend and retract radially, between an open/extended position to a closed/imaging position, about the main axis. The PET detector is coupled to the body of the MRI imager at one of the ends of the MRI bore by any suitable fastening mechanisms. The fastening mechanisms may be realized as mechanical flanges, clamps, bolts, bearings, electromagnets, or any other fixtures that allow holding the PET detector firmly to the MRI imager.

The PET detector system is configured to be movable in one or more of an axial, transaxial, and circumferential directions by means of flange and groove mechanism, guide rails, etc. The movement of the PET detector system is controlled by any suitable electrical and mechanical means such as motors, gears and pinions, spring loads, etc. In an embodiment, the movement of the PET detector system is coordinated with a movement of the object table. Using either a mechanical or electrical transmission means, the PET detector movement is controlled by the movement of the object table or vice-versa. For example, when the object table is advanced towards the PET detector system, a motion sensor or the like senses a movement of the object table and provides a corresponding trigger signal to initiate a movement of the PET detector system between the open position and the imaging position. The speed of extending/retracting the PET detector system may be proportional to the speed of advancing/withdrawing the object table. It is also envisaged that the movement of the PET detector system and object table be purely mechanical where a movement of the object table by manual means translates into a force sufficient to operate the movement of the PET detector system. The PET detector system may further retract to the original position by means of a spring load or so.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A magnetic resonance imager (MRI) and positron emission tomography(PET) imager for performing imaging of an object, the MRI-PET imager comprising:
an MRI bore configured to perform MR imaging of the object, wherein the MRI bore is configured to provide clearance between the MRI bore and the object within the MRI bore; and
a PET detector system coupled to the MRI bore to detect PET emissions from the object, the PET detector comprising at least one detector element retractably arranged to the MRI bore.

2. The MRI-PET imager of claim 1, wherein the PET detector system coupled to the MRI bore is disposed outside the MRI bore

3. The MRI-PET imager of claim 1, wherein the at least one PET detector element is retractably arranged exterior to the MRI bore.

4. The MRI-PET imager of claim 1, wherein the at least one PET detector element defines an aperture for the PET detector system, wherein the aperture size is adjustable through a movement of the at least one detector element.

5. The MRI-PET imager of claim 4, wherein the at least one PET detector element extends to increase the aperture of the PET detector to provide clearance for a first section of the object through the PET detector system into the MRI bore and a second section of the object through the PET detector system.

6. The MRI-PET imager of claim 4, wherein the second section of the object has a larger volume than the first section of the object.

7. The MRI-PET imager of claim 4, wherein the at least one PET detector retracts to decrease the aperture of the PET system to provide optimal clearance between the PET detector system and the object within the aperture.

8. The MRI-PET imager of claim 1, wherein the at least one PET detector element slides between a first position and a second position outside the MRI bore to provide clearance for the object to traverse into the MRI bore.

9. The MRI-PET imager of claim 1, wherein the PET detector system comprises a detector ring formed of at least two segments retractable along a radial axis.

10. The MRI-PET imager of claim 1, wherein the MRI-PET imager is dedicated to imaging a brain.

11. A combined MRI-PET imager comprising:
an MRI bore configured to capture an MR image of an object positioned within the MRI bore; and
a PET detector system disposed outside the MRI bore to detect PET emissions from the object, the PET detector system comprising at least one PET detector element retractably arranged exterior to the MRI bore.

12. The combined MRI-PET imager of claim 11, wherein the at least one detector element is retractable to provide clearance for an object traversing through an aperture defined by the at least one detector.

13. A method of operating a combined MRI-PET imager, the method comprising:
configuring an MRI bore to capture an MR image of an object positioned within the MRI bore; and
disposing a PET detector system outside the MRI bore to detect PET
emissions from the object, wherein the PET detector system comprises at least one detector element retractably arranged exterior to the MRI bore.

14. The method of claim 13, wherein the configuring an MRI bore to capture an MR image comprises sizing the MRI bore to provide optimal clearance between the MRI bore and the object to be imaged.

15. The method of claim 13, further comprising forming the PET detector system comprising at least two segments and configuring the at least two segments to extend along a first direction to provide clearance for the object to traverse into the MRI bore.

16. The method of claim 15, further comprising configuring the at least two detector segments to retract along a second direction to provide optimum clearance between the PET detector and the object for imaging.

17. The method of claim 13, further comprising configuring the MRI-PET imager to control a movement of the object within the imager corresponding to a movement of the PET detector.

18. The method of claim 13, further comprising slidably connecting the at least one PET detector outside the MRI bore, wherein the at least one PET detector slides to provide clearance for the object to traverse into the MRI bore.

19. A method of performing combined MRI-PET imaging of an object in a combined MRI-PET imager, the method comprising:
retracting a detector element of a PET detector system to provide clearance for the object to traverse into an MRI field of view (FOV);
capturing an MR image of the object being positioned into the MRI FOV;
receiving the object within the PET detector FOV;
re-positioning the PET detector element to provide clearance between the PET detector and the object; and
capturing a PET image of the object within the PET detector FOV.

20. The method of claim 19, wherein retracting the PET detector element to provide clearance between the PET detector and the object comprises controlling an aperture defined by the at least one detector element by retracting the detector element.

21. The method of claim 19, wherein receiving the object within a PET detector FOV comprises traversing the object out of the MRI bore and positioning the object within the PET detector FOV.

22. The method of claim 19 further comprising traversing the object through the combined MRI-PET imager in tandem with the extending and retracting of the PET detector element.
